(19) United States
(12) Patent Application Publication
(10) Pub. No.: US 2025/0084404 A1
(43) Pub. Date: Mar. 13, 2025

(54) ENRICHMENT OF GENETIC MATERIAL WITH UPSCALED COT-1 DNA QUANTITIES

(71) Applicant: Helix, Inc., San Mateo, CA (US)

(72) Inventors: Nicole Leonetti, San Diego, CA (US); Eva Lorena Mora-Blanco, San Diego, CA (US)

(21) Appl. No.: 18/244,592

(22) Filed: Sep. 11, 2023

(51) Int. Cl. C12N 15/10

(52) U.S. Cl. CPC C12N 15/1082

(57) ABSTRACT

Various embodiments disclosed relate to a method of processing genetic material for bioinformatic analysis. The present disclosure includes methods of processing genetic material, including receiving a library of genetic material sourced from a sample, adding a large amount of human Cot-1 DNA to the library to produce an enrichment pool, enriching the genetic material in the enrichment pool to produce enriched genetic material, and sequencing the enriched genetic material to produce sequencing data.

### TECHNICAL FIELD

The subject matter disclosed herein generally relates to collection and processing of genetic material. More specifically, the subject matter herein relates to methods of genetic material enrichment for sequencing.

### BACKGROUND

Genetic material can be collected and processed for various forms of bioinformatic analysis. During processing, such genetic material is typically enriched prior to sequencing. During enrichment, a number of probes or assays may be used to capture specific exons or other targeted areas of the genetic material which are desired for sequencing.

A variety of blockers can be used during enrichment to allow for efficient capturing of on-target genetic sequences and efficient subsequent sequencing of genetic material. Human Cot-1 deoxyribonucleic acid (DNA) is one such blocker. Commercial human Cot-1 DNA comprises low complexity regions of DNA isolated from placentas that can be used for prevention of hybridization of repetitive DNA sequences such as Alu and Kpn elements. Cot-1 DNA can be used, for example, to block nonspecific hybridization.

### SUMMARY OF THE DISCLOSURE

The methods discussed herein can be used for large-scale enrichment of human genetic material. These methods can reduce off-bait capture in a manner that facilitates the pooling of many libraries or a large mass of pooled libraries, in a single enrichment process. The method leverages a large amount of Cot-1 DNA, such as in an amount of ten factor or more larger than conventional methods.

In some aspects, the techniques described herein relate to a method of processing genetic material for bioinformatic analysis, the method including: receiving a library of genetic material sourced from a sample; adding human Cot-1 DNA to the library at a ratio of between 5:3 and 20:3 of the human Cot-1 DNA to the genetic material to produce an enrichment pool; enriching the genetic material in the enrichment pool to produce enriched genetic material; and sequencing the enriched genetic material to produce sequencing data.

In some aspects, the techniques described herein relate to a method of processing genetic material for bioinformatic analysis, the method including: receiving a library of genetic material sourced from a sample; adding human Cot-1 DNA to the library to produce an enrichment pool, wherein the human Cot-1 DNA is at least 10 μL of human Cot-1 DNA a concentration of at least 10 mg/mL; enriching the genetic material in the enrichment pool to produce enriched genetic material; and sequencing the enriched genetic material to produce sequencing data.

In some aspects, the techniques described herein relate to a method of processing genetic material for bioinformatic analysis, the method including: receiving a library of genetic material sourced from a sample; adding at least 100 μg human Cot-1 DNA to the library to produce an enrichment pool; enriching the genetic material in the enrichment pool to produce enriched genetic material; and sequencing the enriched genetic material to produce sequencing data.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A depicts a diagram of an example bioinformatics analysis system.

FIG. 1B is a flow chart illustrating an example method of using a bioinformatics analysis system.

FIG. 2A is a flow chart illustrating a method of intaking and preparing a genetic sample in a wet laboratory for bioinformatic analysis in an example.

FIG. 2B is a flow chart illustrating a method of intaking a genetic sample in a wet laboratory for bioinformatic analysis in an example.

FIG. 2C is a flow chart illustrating a method of preparing a genetic sample in a wet laboratory for bioinformatic analysis in an example.

FIG. 3 depicts a method of processing genetic material for bioinformatic analysis, where the enrichment steps use upscaled quantities of Cot-1 DNA in an example.

FIG. 4 is a block diagram of a general-purpose computer that may be programmed into a special purpose computer suitable for implementing one or more examples of the manifest record generating program disclosed herein.

FIG. 5 depicts off-bait capture in a first example.

FIG. 6 depicts off-bait capture in a second example.

FIG. 7 depicts off-bait capture in a third example.

### DETAILED DESCRIPTION

The present disclosure describes, among other things, a method of large-scale enrichment of human genetic material. The methods discussed herein can reduce off-bait capture in a manner that facilitates the pooling of many libraries (e.g., upwards of ten to fifty libraries), or a large mass of pooled libraries, for a single enrichment process. The method leverages a large amount of Cot-1 DNA, at an amount of ten factor or more larger than conventional methods.

The increased amount of Cot-1 DNA allows for an enhanced number of on-target reads (i.e., on-target capture of genetic sequences). This enhances the effective capacity within a flow cell and the number of samples per flow cell used during enrichment. Flow cells, used in sequencing, are consumable items, and typically have a limited number of reads that they can perform. The method increases the number of exomes that can be sequenced using a single flow cell by about 50% to about 70%. In short, by increasing the number of on-target reads and reducing the number of off-target reads, this technique can reduce the number of reads on the flow cell that are wasted in sequencing irrelevant sequences (e.g., junk DNA, bacterial DNA, or other undesired sequences).

In preparation for the enrichment method discussed herein, biological samples can be obtained and accessioned at a wet laboratory. At the wet laboratory, samples can be plated, genetic material can be extracted from the plated samples, and libraries of the genetic material can be prepared.

The method itself can include receiving a prepared library (or multiple libraries that have been pooled together) of genetic material and combining that library with a Cot-1 DNA mixture, in addition to any other appropriate blocker(s). Several probe sets can then be added to the library, including exon probes and/or booster sets of probes that increase coverage of desired genomic regions. The library is then enriched. The range of probes can be tailored as a group to bind to specific alleles, specific genes, the exome, etc. The enrichment process can further include controlling a concentration of the genetic material in each of the wells, and purification and/or elution of the resulting material. After enrichment, the targeted genetic material can be sequenced, such as for use in bioinformatic analysis.

During enrichment, such as hybrid capture, if nonspecific repetitive strands of DNA in the human genome are not blocked, the overall quality of the enrichment process can suffer. For instance, if repetitive strands are not blocked, probes used in the target library might bind to those repetitive strands. This can result in “off-bait” capture of genetic material by the probes, wherein undesired genetic material, such as those repetitive strands, is captured. Additionally, off-bait capture can lead to amplification of these off-target regions, resulting in a ratiometric reduction of target regions. This can reduce coverage of desired regions and wasted sequence reads. For example, if off-target genetic material is amplified, then corresponding sequencing reads will not fall within the targeted regions. This can result in lower overall coverage of the targeted regions, particularly when a static number of reads is performed as a part of sequencing.

Blockers can be used to prevent off-target capture, such as to block repetitive strands that are not of interest. For example, Cot-1 DNA can be used to block nonspecific hybridization by binding repetitive sequences of DNA. Cot-1 DNA includes placental DNA enriched with repetitive sequences of about 50 to 100 base pairs. Cot-1 DNA helps block nonspecific hybridization by binding repetitive sequences of DNA. Use of Cot-1 DNA during enrichment to bind repetitive sequences can prevent hybridization of those sequences by probes, thus preventing amplification of those sequences.

Suppliers of Cot-1 DNA instruct that a small amount of Cot-1 DNA must be used, such as a small volume at a concentration as low as 1 mg/mL. Use of larger amounts of Cot-1 DNA has been studied but causes distortion of quantitative genomic and expression hybridization.

When the instructed amount of Cot-1 DNA is used, a limited number or mass of libraries can be used during enrichment. Total library mass is the mass of all libraries pooled for enrichment, and includes sheared genomic DNA with adaptors that have been appended and ligated on their ends. Thus, total library mass can be the combined mass of DNA and bound probes used as input to an enrichment process. For example, in an embodiment where four uniquely indexed libraries of 250 ng (each corresponding to a different sample) are pooled, the total library mass for enrichment will be 1 μg of DNA

If a larger number of libraries or a larger total library mass is used with the instructed amount of Cot-1 DNA, more off-bait capture occurs, reducing overall quality. This can occur because the small amount of Cot-1 DNA competes with probes within each library to bind to repetitive sequences of DNA. As more libraries are pooled for enrichment, Cot-1 DNA competes with a larger number of probes to bind to repetitive sequences. This reduces the efficacy of Cot-1 DNA in blocking those probes from binding to repetitive sequences.

The methods discussed herein leverage a highly upscaled amount of Cot-1 DNA relative to the total library mass to increase on-target capture of sequences, permitting the pooling of a large number of libraries for enrichment. The use of highly upscaled Cot-1 DNA (e.g., by a factor of ten or more) can allow for enhanced breadth of coverage during sequencing without sacrificing quality, by reducing off-target captures (e.g., junk DNA or bacterial DNA). In short, using highly upscaled Cot-1 DNA permits pooling of additional libraries in a single enrichment process, because the Cot-1 DNA becomes more effective as a blocker, enhancing the efficacy of the enrichment process.

The methods discussed herein include use of a mass of Cot-1 DNA which, when applied at a standard concentration of 1 mg/ml, may have a larger volume than the capacity of a standard microplate well. The methods discussed herein may also include larger volumes of Cot-1 DNA than those anticipated by commercially available sequencing kits. In some examples, the methods discussed herein maintain Cot-1 DNA volume, but increase the Cot-1 DNA concentration significantly, such as from about 1 mg/mL to a concentration of about 10 mg/mL.

The advantages of using highly upscaled Cot-1 DNA are surprising and unexpected compared to existing uses and studies of Cot-1 DNA. For example, studies suggest that Cot-1 DNA enhances non-specific hybridization between probes and genomic targets. Hence, highly upscaled Cot-1 DNA is expected to interfere with the ability of probes to bind with targets during enrichment, resulting in increased off-bait capture. In another study, it was seen that Cot-1 DNA contained sequences that competed with labeled targets for probe sites. (See, for example, “Distortion of quantitative genomic and expression hybridization by Cot-1 DNA: mitigation of this effect”, Newkirk et al., Nucleic Acids Research, Volume 33, Issue 22, 1 Dec. 2005, Page e191). In either case, an increased amount of Cot-1 DNA relative to the standard levels discussed above has been expected to increase competition with probes, increase off-bait capture, and reduce sequencing quality. However, as discussed and shown herein, a large upscaling of Cot-1 DNA has the opposite effect of increasing on-target captures.

### Definitions

As used herein, “accession”, or “accessioning” refers to receiving and preparing a sample for later laboratory processes.

As used herein, “amplifying” refers to the production of multiple copies of a sequence of nucleic acid or other genetic material, such as RNA or DNA.

As used herein, “bioinformatics” refers to the science of collecting complex biological data such as genetic codes.

As used herein, “biological sample”, or “sample” refers to a specimen from a patient, such as for bioinformatic research.

As used herein, “contamination” refers to a sample that is impure, polluted, or unsuitable for biological analysis and research.

As used herein, “genetic material” refers to a fragment, molecule, or a group of nucleic acids, such as DNA or RNA, genetic material from one or more chromosomes, mitochondrial genetic material, or other genetic material.

As used herein, “mutation” refers to a changed structure of a gene that results in a variant form of the gene (e.g., with respect to a reference genome).

As used herein, “pathogen” refers to a bacterium, virus, or other microorganism that can cause disease.

As used herein, “PCR” refers to quantitative polymerase chain reaction used for measuring DNA via polymerase chain reaction amplification techniques.

As used herein, “sequencing” refers to a process of determining the nucleic acid sequence, and the order of nucleotides in genetic material.

As used herein, “variant” or “genetic variant” refers to a subtype of a microorganism that is genetically distinct from other subtypes.

### Bioinformatic Analysis System and Methods

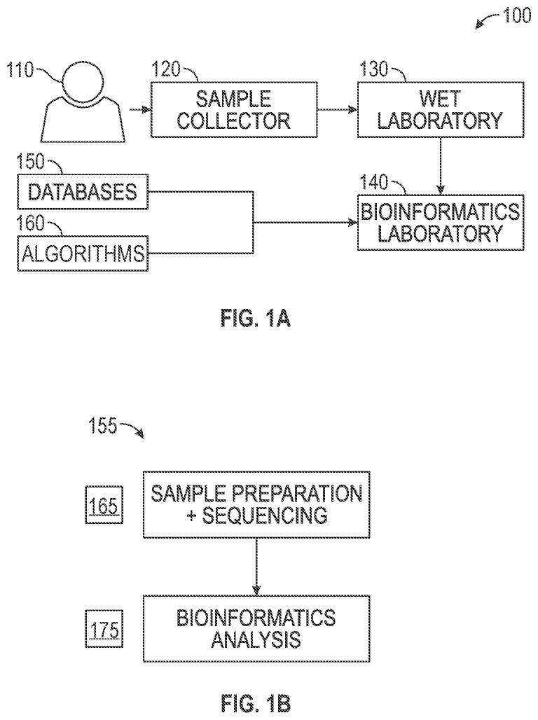

FIG. 1A depicts a diagram of an example bioinformatics analysis system 100, while FIG. 1B is a flow chart illustrating a method 155 of using such a bioinformatics analysis system 100.

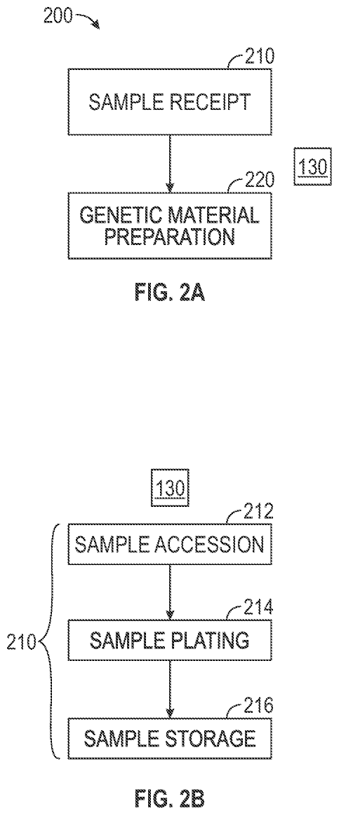

The system 100 can include both physical or “wet” laboratory components, and bioinformatics components. For example, the system 100 can interact with patients 110, from whom biological samples can be collected, in addition to sample collectors 120, which may be, for example, doctors, pharmacies, or other appropriate entities that can acquire patient samples. The system 100 includes a wet laboratory 130 which is positioned to receive the biological samples and process those samples to produce sequenced genetic material for analysis, such as at step 165 of method 155. These methods of sample receipt, handling (e.g., accessioning), and sequencing, are discussed in detail below with reference to FIGS. 2A to 2C.

The system 100 can additionally include data driven components, such as databases 150 and algorithms 160 or other programs that support a bioinformatic laboratory 140 used to analyze genetic information. These data driven components can be used to do bioinformatic analysis (step 175 in method 155).

### Sample Processing Methodology

Before bioinformatic analysis, biological samples are collected and sequenced through physical components of the system 100, such as through the wet laboratory 130. Methods of receiving and processing such samples are summarized in FIGS. 2A to 2C. FIG. 2A is a flow chart illustrating a method 200 of intaking and preparing a sample for sequencing in a wet laboratory for bioinformatic analysis. The method 200 can include two primary portions: receiving the samples (step 210) and preparing genetic material from the samples (step 220). FIG. 2B illustrates portions of step 210, including a method of intaking a sample for sequencing in a wet laboratory for bioinformatic analysis. FIG. 2C illustrates portions of step 220 of the method 200, a method of preparing a sample in a wet laboratory for bioinformatic analysis.

The method 200 can begin with sample collection. For example, the samples can be collected by receiving a nasal swab, blood, saliva, or other material potentially containing genetic material of interest.

Accessioning Samples. Once received at the laboratory, at step 212, the samples can be accessioned, that is, prepared for later laboratory processes. For example, accessioning can include receiving a batch of samples. A batch of samples can include, for example, hundreds of individual samples, or thousands of individual samples. Each sample can be retained in a sample container. For example, test tubes can be used to store each of the samples. The sample containers can be sealed to help prevent environmental exposure and prevent sample co-mingling. For example, the sample containers may be sealed via a cap that is threaded, glued, press-fit, or otherwise affixed via appropriate sealing mechanism. When the samples are received in a batch, the corresponding sample containers may also include one or more remnants of a sampling tool, such as a swab used to collect the sample.

In some cases, the sample containers may be accompanied by Customer Sample Identifiers (CSI) such as by a component affixed to or integrated with the sample container. Such a CSI can uniquely distinguish individual sample containers from other sample containers being received. For example, a CSI may uniquely distinguish a sample from other samples in the same batch, other samples received on the same date, or other samples received from the same customer. Such CSI can be provided as a label such as a bar code or a Quick Response (QR) code, a chip such as a Radio Frequency Identifier (RFID), or another type of visual, transmission-generating, or other component affixed to or integrated with the sample container.

In some cases, the sample containers can be further sealed in an external container, such as a bag. External containers can help prevent contamination of samples, such as by preventing biological material from the samples contacting other or external surfaces. An external container can also help prevent cross-contamination between samples. Moreover, when a sample includes blood or a pathogen, the external container can provide an additional barrier to protect technicians who may handle the samples. The external container can additionally include documentation correlating to the CSI, such as information on the patient that the sample was sourced from, information indicating circumstances of sampling, for example, a sampling date, a sampling method, a location that the sample was acquired, a name or title for a person who performed the sampling, other information, or combinations thereof.

In some cases, the samples can be in a chemical solution. For example, the sample may be prepared in an aqueous solution, such as a saline solution. In some cases, the samples can include a bodily fluid such as saliva, mucus, blood, or other. In an example, the sample can have a volume of about 2 mL, of about 3 mL, of about 4 mL, or of about 5 mL.

The samples include genetic material. For example, the samples can include Deoxyribonucleic Acid (DNA) or Ribonucleic Acid (RNA). In an example, the genetic material is one or more of many constituent components within the sample. For example, one portion of the genetic material may exist within the nuclei or mitochondria of white blood cells that are included within the sample. In another example, another portion of the genetic material may exist within viruses or bacteria within the sample. In these types of examples, the genetic material is not yet isolated from the remaining constituent components of the sample. Thus, the genetic material should be isolated.

To begin isolating the genetic material, batches of the samples can be heated in ovens to facilitate cell lysis. The temperature and duration of heating can be chosen such that any pathogenic material within the samples is rendered harmless, such that cellular lysis occurs, or both. For example, the samples can be heated at a temperature of between about 40° C. and 80° C., or at a temperature of between about 15° C. and 200° C., or at another appropriate temperature range. The samples can be heated for a time period of about 30 minutes, or for a time period of about 50 minutes, or for another appropriate time period. In some cases, such as where the samples are the contents of a blood draw, the heating step may be skipped.

After heating, the batches of samples can be removed from the ovens. In an example, sample containers can be removed from external containers, such as by cutting open the external containers. The sample containers can be inspected, either in a manual, automated, or semi-automated fashion. For example, a technician or an automated system can determine the CSI for the sample and compare the CSI to documentation accompanying the batch. If there is a discrepancy between the CSIs on the sample container and in the documentation, the sample may be flagged as having an error condition. Similarly, if the CSI on the sample container is damaged (such as by abrasion, heat-damage, or water-damage) and has become unreadable, the sample may be flagged as having an error condition.

In some cases, the technician or automated system can further inspect the contents of the sample container, such as visually. If the sample does not include expected constituent components, then the sample can be flagged as having an error condition. For example, if the sample includes a fluid that is not permitted (such as extraneous blood), includes an entire swab or no swab, is within a fractured or broken sample container, or is outside of an expected range of volume (e.g., between two and five milliliters), or other conditions, then the sample can be flagged as having an error condition.

Subsequently, samples that have not been flagged with an error condition can proceed to sample integration. Here, the sample can be assigned a Laboratory Sample Identifier (LSI). Such an LSI can uniquely identify the sample from other samples received in the same batch, received on the same day, processed in the same laboratory, handled by the same company for sequencing, or combinations thereof. The LSI can be stored in a laboratory sample database, and uniquely correlated to the CSI for the sample. The LSI can be associated with any error codes reported from the sample. Both the CSI and the LSI can both be applied to the sample container.

Sample Plating. Once accessioned, the samples can be plated at step 214. At this point, the samples have been successfully integrated into the laboratory environment and are ready for analytics. The samples can next be prepared for transfer to a sample microplate. The sample microplate can be labeled with a unique identifier, which can distinguish the sample microplate from other sample microplates. For example, the sample microplate can be a solid body with about 50 wells to about 400 wells, distributed across rows and columns, each well having a capacity of about 30 μL to about 300 μL. In other examples, different size microplates with a different number of wells at varying volumes can be used.

The samples to be used on the microplate may be racked and the rack may be assigned an identifier, such as to allow a technician to understand which samples correspond to which LSIs. The technician may unseal the sample, such as by a manual, automated, or semi-automated tool to efficiently open the sample container. The tooling may, for example, unscrew, cut, or drill each sample container, to make the sample within available for physical transfer to the sample microplate.

The samples can then be transferred to the microplate, such as by an automated robot that operates an end effector in accordance with one or more programs for effective transfer of the samples. This can be done, for example, with a combination of actuators, piezoelectric elements, pressure systems, and/or other components operating the end effector of the robot. The end effector can uptake portions of the samples in micropipettes and transfer those samples to the corresponding wells in the microplate. In some cases, disposable tips can be used. In some cases, portions of the samples can be transferred. In some cases, reagents can be added to the samples. In some cases, controls can be included in the microplate. The sample microplate, once completed, can be transferred for further processing in the laboratory.

Sample Storage. After plating, the samples can be stored at step 216. In some cases, accessioned samples, plated samples, or other samples, are stored for later use. In this case, they can be stored at room temperature, or can be cryogenically frozen and arranged on racks for later retrieval. Samples can be preserved for periods of days or years to allow later rapid re-testing.

Extraction of Genetic Material. When genetic analysis is desired, the genetic material of the samples can be extracted for sequencing at step 222. In some examples, a reagent can be applied to sample wells to lyse cells therein to expose genetic material.

Additionally, aspirating, and dispensing reagents can be used to selectively bind genetic material released from lysed cells. In some examples, this can include applying a bead to the well. In this case, the beads can, for example, be magnetic beads that selectively bind to the genetic material. This can help allow for isolation and purification of the genetic material at the bead, leaving contaminants in the solution. In an example, a magnetic bead can be magnetically drawn to a magnetic base at or under the sample microplate. In this case, after the genetic material has been drawn to the bead, a flushing step can be performed to wash away remaining fluid, helping to remove impurities.

In some examples, fluid can be added or removed from wells, such as to concentrate or elute the genetic material. Fluid can be transferred from the wells of the sample microplate to a genome stock microplate. In an example, a portion of fluid can be removed from each well for quality control purposes. This can, for example, be used to determine concentration of genetic material therein.

Library Preparation. After extraction of the genetic material, a library can be prepared using the contents of the genome stock microplate at step 224. For example, the bead for each well, including ionically bonded genetic material, can be transferred to a distinct well of a library preparation microplate. The library preparation microplate can include an identifier. The LSI associated with each well on the sample microplate can be mapped to a corresponding well on the library preparation microplate. The library preparation microplate may be transferred to a new portion of the laboratory to help prevent amplified genetic material from entering portions of the laboratory where genetic material has not been amplified, which could result in contamination.

A reagent can be applied to each well of the library preparation microplate. The reagent can ionically bond to the surface of the bead within the well more strongly than the genetic material. This helps release the genetic material from the surface of the bead of each well, enabling the genetic material to be chemically interacted with.

Library preparation can include normalization of a concentration of genetic material in each well of the sample microplate. Library preparation can further include fragmentation of the genetic material via an enzyme or via the application of physical forces. During this process, the entire genome (e.g., roughly three billion base pairs for a human genome), may be fragmented into pieces. In an example, the pieces can be about 300 to 400 base pairs in length. These pieces can be referred to as nucleic acid fragments. These nucleic acid fragments can undergo adaptor ligation and indexing. In an example, this can include Next Generation Sequencing (NGS) library preparation processes.

The genetic material can then be amplified, such as by Polymerase Chain Reaction (PCR) amplification. The resulting solution can be purified and eluted. During this library preparation, one or more reference samples of genetic material can be added to the wells of the library preparation microplate. The reference samples can serve as controls and aid in quality control.

Once the library preparation has been completed, thousands or millions of distinct fragments of the genetic material, each corresponding with a different portion of a genome of the subject, can be ligated to predefined adapters that bind with the genetic material. Each of the adaptor ligated fragments is referred to as a “library.”

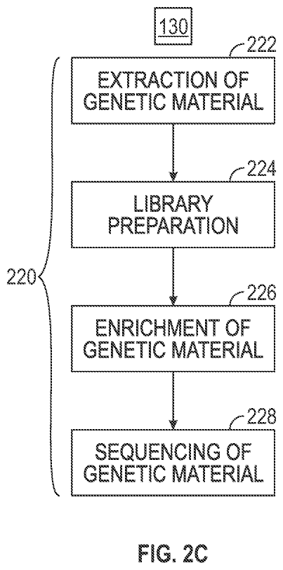

In the methods discussed herein, multiple libraries can be pooled together for the enrichment process as discussed below. For example, once prepared, several libraries can be pooled, such as up to 5, 10, 15, 20, 25, 30, 35, 40, 45, or 50 libraries. For example, twelve uniquely indexed libraries of 250 ng each, which each correspond to a different sample, can be pooled prior to enrichment, resulting in a total library mass of 3 μg. A specific example of using upscaled Cot-1 DNA with enrichment of multiple pooled libraries is shown and discussed below with reference to FIG. 3.

In additional examples, probes applied to each well can include chemical identifiers (“barcodes”) that are distinct from each other. The use of a different chemical identifier for probes applied to each well of the well plate can enable sequencing to later be performed for multiple subjects on the same flow cell, without conflating sequencing results for those subjects.

In additional examples, the library preparation process can further include controlling a concentration of the genetic material in each well, and purification and/or elution of the resulting material. Similar to the processes performed after extraction of genetic material, concentration of genetic material after library preparation can be confirmed for each well via testing.

Enrichment of Genetic Material. After library preparation, enrichment processes can be performed in order to either directly amplify (e.g., via amplicon or multiplexed PCR) or capture (e.g., via hybrid capture) predefined libraries of genetic material, such as at step 226 in FIG. 2C. This can enhance the ease of sequencing desired portions of the genome.

Here, probes can be used during genetic sample enrichment, prior to amplification, to capture targeted genetic material. In some cases, pathogen genetic material can also be captured, via the use of pathogen-specific probes, such as in a viral assay. The captured genetic material is amplified and sequenced.

In the methods discussed herein, Cot-1 DNA can be used during this enrichment step as a blocker. Generally, Cot-1 DNA is used to block nonspecific hybridization in microarray screening, and to suppress repetitive DNA sequences. As discussed herein, Cot-1 DNA can be used in upscaled quantities during enrichment to allow for increased on-target analysis during sequencing. A specific example of using upscaled Cot-1 DNA with enrichment of multiple libraries is shown and discussed below with reference to FIG. 3.

In an example, during enrichment, customized biotinylated oligonucleotide probes can be applied to the libraries. The probes can selectively hybridize genetic material occupying desired portions of the genome for the genetic material, such as specific genes, or the entire exome. Magnetic beads can bind to biotin molecules in the probes to attach the hybridized material to the magnetic beads. Magnetic forces can capture the beads in place, enabling remaining fluid within each well to be removed or washed out, thereby removing impurities, and leaving only the genetic material that is desired. Thus, genetic material can be released from the beads in a similar manner to that discussed above for prior processes.

In an example, hybrid capture target enrichment can be performed. During this process, the probes can include tailored oligonucleotides that are chosen to bind to the genetic material. The range of probes can be tailored as a group to bind to specific alleles, specific genes, the exome, the entire genome, etc. That is, each probe can bind to a nucleic acid fragment at a specific location on the genome, and the range of probes can be selected to ensure that alleles, genes, the exome, or the entire genome of the subject being considered is acquired. In examples where probes are targeted to a portion of the entire genome, efficiency of the sequencing process is enhanced, by foregoing the need to sequence all of the roughly three billion base pairs found in the human genome.

The enrichment process can further include controlling a concentration of the genetic material in each well, and purification and/or elution of the resulting material. Similar to the processes performed after extraction of genetic material, concentration of genetic material after enrichment can be confirmed for each well via testing.

Sequencing of Genetic Material. After enrichment, the genetic material can be sequenced at step 228. Sequencing can be performed according to any of a variety of techniques, including short-read and long-read techniques.

In an example, the sequencing can be performed as Sequencing by Synthesis (SBS) at genetic analyzer equipment. For example, sets of enriched libraries of genetic material bound to probes in earlier steps can be transferred to a flow cell, and annealed to oligonucleotide probes within the flow cell. At this stage, the contents of multiple wells can be applied to the same flow cell, because the libraries within those wells are tagged with the chemical identifiers referred to above.

In an example, the chemical identifiers can include nucleotide sequences that are detectable during the sequencing process to determine a corresponding LSI. Complementary sequences can then be created via enzymatic extension to create a double-stranded portion of genetic material. The double-stranded genetic material can then be denatured, and the portion of the genetic material consisting of the library fragment can be washed away. Bridge amplification can then be performed to create copies of the remaining molecule in a localized cluster. For example, a cluster can comprise twenty to fifty copies of the same molecule, localized to a location the size smaller than a pinhead on the flow cell. Sequencing primers can be annealed to library adapters to prepare the flow cell for SBS. During SBS, the sequencing primer uses reverse terminator fluorescent oligonucleotides, one base per cycle, for several cycles in the forward direction. After the addition of each nucleotide, clusters can be excited by a light source, resulting in fluorescence which can be measured. The emission wavelength and signal intensity for each cluster determines a base call for that cluster. A chemical group blocking a 3′ end of the fragment can then be removed, enabling a subsequent nucleotide to be read. This can help control nucleotide addition and detection. After each cycle, denaturing and annealing can be performed to extend the index primer. A complementary reverse strand can be created and extended via bridge amplification. The reverse strand can then be read in the reverse direction for a number of cycles, in a manner similar to reads in the forward direction.

Throughout the processes discussed above, the laboratory environment can be carefully controlled to ensure quality. For example, temperature within each segment of the laboratory can be carefully monitored and controlled, and ultraviolet lighting or other features capable of inactivating genetic material can be carefully positioned to ensure that contamination does not occur.

In general, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text based FASTQ format, that reports quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics such as quality indicator metrics. The called sequence data may be provided to a data analyst via a User Interface (UI), such as a Graphical User Interface (GUI) presented via a display. The technician may then validate the resulting called sequence data and release it for reporting to subjects, health care providers, and/or scientists.

Enrichment with Upscaled Cot-1 DNA

FIG. 3 depicts a method 300 of processing genetic material for bioinformatic analysis, where the enrichment steps uses upscaled quantities of Cot-1 DNA. The method 300 can include blocks 310 to 340.

At block 310, a library of genetic material is received. The library of genetic material can be created from genetic material from a biological sample that has been accessioned and prepared as discussed above with reference to FIGS. 2A to 2C. In many embodiments, the library is pooled with other libraries for other biological samples, resulting in a set of pooled libraries for enrichment.

A large number of pooled libraries can be received together for enrichment through the example method 300. In an example, more than two libraries can be pooled. In an example, more than 5 libraries, more than 10 libraries, more than 15 libraries, more than 20 libraries, more than 25 libraries, more than 30 libraries, more than 35 libraries, more than 40 libraries, more than 45 libraries, or more than 50 libraries can be pooled. In an example, between ten to fifty libraries can be received. In an example, twelve libraries can be received.

These libraries can be quality controlled and indexed, such as described above with reference to FIGS. 2A to 2C. The libraries can then be pooled after being indexed and prior to enrichment. Individual libraries can each have a mass of about 100 to 500 ng DNA (e.g., 250 ng DNA). In an example, a large total library mass of the pooled libraries can be used, such as larger than about 1 μg, larger than about 2 μg, larger than about 3 μg, larger than about 4 μg, or larger than about 5 μg.

The receipt and enrichment of a large number of pooled libraries simultaneously helps increase sequencing quality and sequencing breadth. This can allow for an overall broader review of the genetic material, such as by allowing enrichment and sequencing of a larger portion of the genome for analysis and review.

At block 320, human Cot-1 DNA and probes can be added to the pooled libraries in the enrichment pool. The Cot-1 DNA can be added by any suitable method, such as by stirring or mixing manually or with appropriate automated tools. Here, the Cot-1 DNA can be added at an upscaled amount. In an example, the Cot-1 DNA can be added to the enrichment pool at a ratio of between 100:1 and 20:1 (e.g., 30:1) of Cot-1 DNA to genetic material by weight.

In an example, Cot-1 DNA can be added to the enrichment pool at a concentration of more than about 8 mg/mL, more than about 9 mg/mL, more than about 10 mg/mL, more than about 11 mg/mL, or more than about 12 mg/mL, such as at a concentration of 10 mg/mL. The aforementioned values refer to concentrations of Cot-1 DNA in-solution, prior to being added to the enrichment reaction. In an example, at least 10 μL of Cot-1 DNA can be added to the enrichment pool, or at least 11 μL, at least 12 μL, at least 13 μL, at least 14 μL, or at least 15 μL of Cot-1 DNA. In an example, Cot-1 DNA can be used at an amount of five to ten times the recommended amount in commercially available kits. Cot-1 DNA can be used in this amount in each of the wells being enriched.

The use of such larger-than recommended quantities of Cot-1 DNA, by helping to reduce off-bait capture, can help to ensure deep coverage and highly efficient use of sequencing consumables such as flow cells. The large amount of Cot-1 DNA here can has been upscaled to reduce off-bait capture, which accommodates the larger number of libraries being enriched together.

The Cot-1 DNA is provided in the wells to perform blocking while the libraries are enriched. Cot-1 DNA is provided at a massively increased amount, such as more than five to ten times greater than recommended amounts found in commercial kits. That is, for a given reaction volume (e.g., 5 μL), an amount of added Cot-1 DNA for that reaction volume may comprise five to ten times more Cot-1 DNA by mass than instructed in commercial kits. To accommodate such an amount of Cot-1 DNA within the limited reaction of a microplate well, a Cot-1 DNA can be used at a greater concentration than in other formats. For example, a concentration of about 10 mg/mL can be used compared to a conventional 1 mg/mL. This may result in the concentration of Cot-1 DNA within the reaction volume (i.e., after mixing with other reagents) to remain five to ten times greater than instructed.

At block 330, the genetic material in the enrichment pool can be enriched to produce enriched genetic material. Example enrichment methods are discussed above with reference to FIG. 2C.

Enriching of the genetic material in the enrichment pool can include capturing targeted portions of the genetic material, such as through the use of probes. For example, probes can be inserted into the enrichment pool, and allow for collection of targeted portions of the genetic material with those probes. Such probes can be exon probes configured to target an exome of a subject that provided the sample. In an example, the probes can include booster probes configured to boost one or more regions of interest in the genetic material, such as multiple sets of booster probes. Probes can be used at different concentrations as desired. Different assays can be used to target specific genetic material.

In an example, enriching the genetic material in the enrichment pool can include amplifying the targeted genetic material prior to sequencing. In an example, enrichment can further include controlling concentration of the targeted genetic material therein, such as by elution.

At step 340, the enriched material can be sequenced to produce sequencing data. In an example, sequencing of the enriched genetic material can include applying the enriched genetic material to one or more flow cells of a sequencing device. In an example, sequencing of the enriched genetic material can include enriching genetic material within a well, where that well has received an increased amount of human Cot-1 DNA (e.g., as a blocker). This can be compared to enrichment pools that are prepared with a standard amount of human Cot-1 DNA. In an example, the library can include genes that cover an entire exome of a subject that provided the sample.

This method notably reduces the amount of “off-bait” capture for DNA sequences. This beneficially enhances sequencing quality. This is advantageous in comparison to previous uses of Cot-1 DNA, which emphasized using a small amount of Cot-1 DNA to avoid experimental noise.

The increase in overall Cot-1 DNA used for enrichment additionally provides an advantage because the amount of patient DNA within each enrichment remains consistent, such as about 200-300 ng or 240-260 ng per uniquely indexed library for a sample. Because a smaller amount of Cot-1 DNA was known to be effective as a blocker on this amount of DNA, there was no expectation that a larger amount of Cot-1 DNA would have an impact of efficacy—but it does, as discussed in the Examples below.

In an example, capturing of the targeted genetic material produces at least 70%, at least 75%, at least 80%, at least 85%, at least 90%, at least 95%, at least 96%, 97%, 98%, or at least 99% on target capture of sequences (e.g., of sequences captured by the probes constitute a portion of an exome of a subject that provided the sample). In an example, capturing of the targeted genetic material produces less than 30%, less than 25%, less than 20%, less than 15%, less than 10%, less than 5%, less than 4%, 3%, 2%, or 1% off-target capture of sequences.

### Computer Example

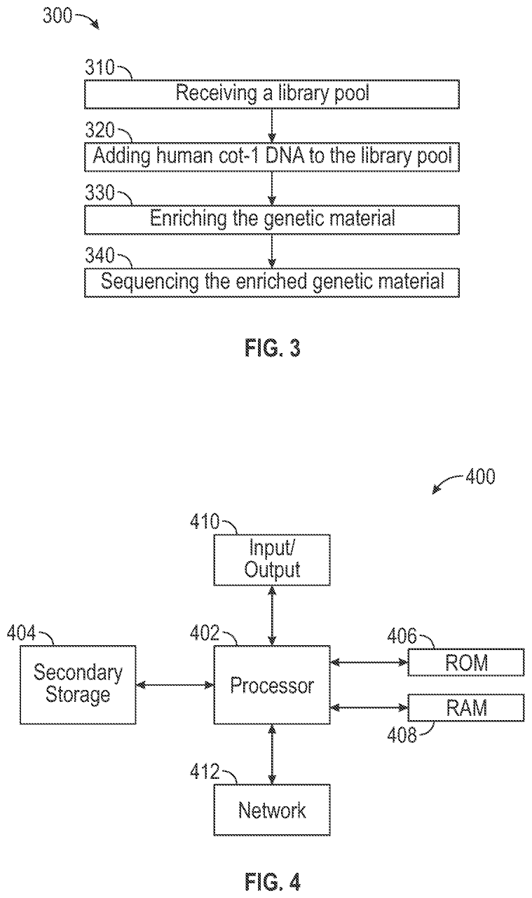

FIG. 4 is a block diagram of a typical, general-purpose computer 400 that may be programmed into a special purpose computer suitable for implementing one or more examples of the manifest record generating program disclosed herein. The manifest record generating program described above may be implemented on any general-purpose processing component, such as a computer with sufficient processing power, memory resources, and communications throughput capability to handle the necessary workload placed upon it. The computer 400 includes a processor 402 (which may be referred to as a central processor unit or CPU) that is in communication with memory devices including secondary storage 404, read only memory (ROM) 406, random access memory (RAM) 408, input/output (I/O) devices 410, and network connectivity devices 412. The processor 402 may be implemented as one or more CPU chips or may be part of one or more application specific integrated circuits (ASICs).

The secondary storage 404 is typically comprised of one or more disk drives or tape drives and is used for non-volatile storage of data and as an over-flow data storage device if RAM 408 is not large enough to hold all working data. Secondary storage 404 may be used to store programs that are loaded into RAM 408 when such programs are selected for execution. The ROM 406 is used to store instructions and perhaps data that are read during program execution. ROM 406 is a non-volatile memory device that typically has a small memory capacity relative to the larger memory capacity of secondary storage 404. The RAM 408 is used to store volatile data and perhaps to store instructions. Access to both ROM 406 and RAM 408 is typically faster than to secondary storage 404.

The devices described herein may be configured to include computer-readable non-transitory media storing computer readable instructions and one or more processors coupled to the memory, and when executing the computer readable instructions configure the computer 400 to perform method steps and operations described above with reference to FIG. 3 to FIG. 4. The computer-readable non-transitory media includes all types of computer readable media, including magnetic storage media, optical storage media, flash media and solid-state storage media.

It should be further understood that software including one or more computer-executable instructions that facilitate processing and operations as described above with reference to any one or all of steps of the disclosure may be installed in and sold with one or more servers and/or one or more routers and/or one or more devices within consumer and/or producer domains consistent with the disclosure. Alternatively, the software may be obtained and loaded into one or more servers and/or one or more routers and/or one or more devices within consumer and/or producer domains consistent with the disclosure, including obtaining the software through physical medium or distribution system, including, for example, from a server owned by the software creator or from a server not owned but used by the software creator. The software may be stored on a server for distribution over the Internet, for example.

Also, it will be understood by one skilled in the art that this disclosure is not limited in its application to the details of construction and the arrangement of components set forth in the description or illustrated in the drawings. The examples herein are capable of other examples, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated examples may be implemented, at least in part, in digital electronic circuitry, analog electronic circuitry, or in computer hardware, firmware, software, or in combinations of them. These components may be implemented, for example, as a computing program product such as a computing program, program code or computer instructions tangibly embodied in an information carrier, or in a machine-readable storage device, for execution by, or to control the operation of, data processing apparatus such as a programmable processor, a computer, or multiple computers.

A computing program may be written in any form of programming language, including compiled or interpreted languages, and it may be deployed in any form, including as a stand-alone program or as a module, component, subroutine, or other unit suitable for use in a computing environment. A computing program may be deployed to be executed on one computer or on multiple computers at one site or distributed across multiple sites and interconnected by a communication network. Also, functional programs, codes, and code segments for accomplishing the techniques described herein may be easily construed as within the scope of the present disclosure by programmers skilled in the art. Method steps associated with the illustrative examples may be performed by one or more programmable processors executing a computing program, code or instructions to perform functions (e.g., by operating on input data and/or generating an output). Method steps may also be performed by, and apparatus may be implemented as, special purpose logic circuitry, e.g., an FPGA (field programmable gate array) or an ASIC (application-specific integrated circuit), for example.

The various illustrative logical blocks, modules, and circuits described in connection with the examples disclosed herein may be implemented or performed with a general-purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computing program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computing program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory may be supplemented by or incorporated in special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill in the art further appreciate that the various illustrative logical blocks, modules, circuits, and algorithm steps described in connection with the examples disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the disclosure. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor may read information from, and write information to, the storage medium. In the alternative, the storage medium may be integral to the processor. In other words, the processor and the storage medium may reside in an integrated circuit or be implemented as discrete components.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store processor instructions. The term “machine-readable medium” shall also be taken to include any medium, or combination of multiple media, that is capable of storing instructions for execution by one or more processors, such that the instructions, when executed by one or more processors cause the one or more processors to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” as used herein excludes signals per se.

### EXAMPLES

Various examples of the present disclosure can be better understood by reference to the following Examples which are offered by way of illustration. The present disclosure is not limited to the Examples given herein.

### Example 1. Off-Bait Capture with Increased Cot-1 DNA

Example 1 illustrates the benefits of using dramatically increased Cot-1 DNA in enrichment. The Example illustrates that this significantly decreased off-bait coverage.

A set of biological samples were prepared through the accession, plating, and library preparation as discussed above. Then, twelve libraries were pooled to an amount of about 3,000 ng (3 μg) total DNA for sequencing. The enrichment pool additionally included biotinylated oligo probes targeting exomes and additional regions of interest (e.g., areas of interest ranging at about 120 base pairs), and an adapter blocking reagent. Each prepared sample had varying amounts of Cot-1 DNA as summarized in the below Table 1, wherein 1× Cot-1 DNA corresponded with 5 μg:

TABLE 1
Samples with varying Cot-1 DNA amounts.

| | Cot-1 DNA Amount |
|---|---|
| Sample 1 | 0.5x |
| Sample 2 | 2x |
| Sample 3 | 20x |

In this Example, enrichment was performed with a commercially available enrichment kit. The hybridization reaction was incubated overnight at a hybridization temperature of about 55 to 65° C. The targets annealed to the biotinylated probes were captured using streptavidin beads. A series of washes were used to remove unbound targets. Then PCR amplification of captured targeted libraries was done.

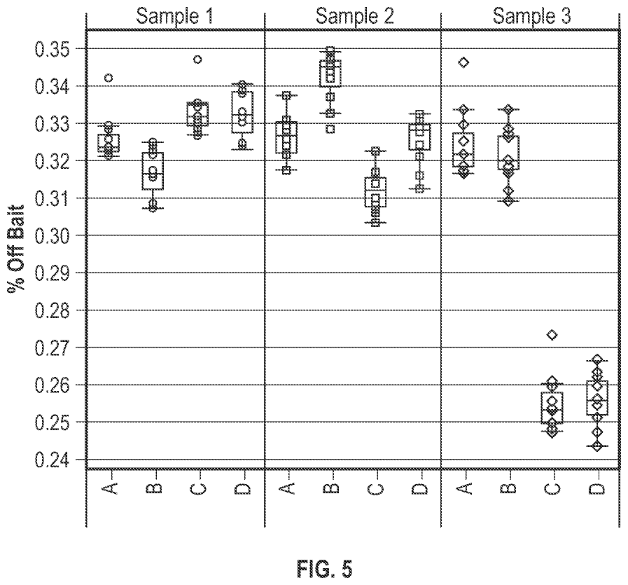

The off-bait capture was recorded from each Sample as shown in FIG. 5. Here, the percent of off-bait capture is shown for two control samples (A, B) and two experimental samples (C, D). Notably, Sample 2, which used four times the amount of Cot-1 DNA with respect to Sample 1, had comparable and only marginally better off-bait capture than Sample 1. This is consistent with the studies cited above, which suggested that increasing the amount of Cot-1 DNA above conventional levels would not be beneficial, and could even be detrimental to the enrichment process. Considered relative to Sample 1, results to Sample 2 suggests that even notable increases in the amount of Cot-1 DNA have no notable impact on off-bait capture.

Surprisingly, these findings did not persist for Sample 3 when compared to Sample 1 and Sample 2. On average, the Sample 1 and Sample 2, which contained 0.5 times and 2.0 times the commercially recommended Cot-1 DNA amount, had substantially more off-bait capture compared to Sample 3, made with 20 times the commercially recommended Cot-1 DNA amount.

### Example 2. Increased Cot-1 DNA

In Example 2, larger amounts of Cot-1 DNA were used compared to Example 1 above. The Samples here are summarized in Table 2 below:

TABLE 2
Samples with increased Cot-1 DNA.

| | Cot-1 DNA Amount |
|---|---|
| Sample 4 (Control) | 1x |
| Sample 5 | 20x |
| Sample 6 | 40x |
| Sample 7 | 20x *reduced blocker |

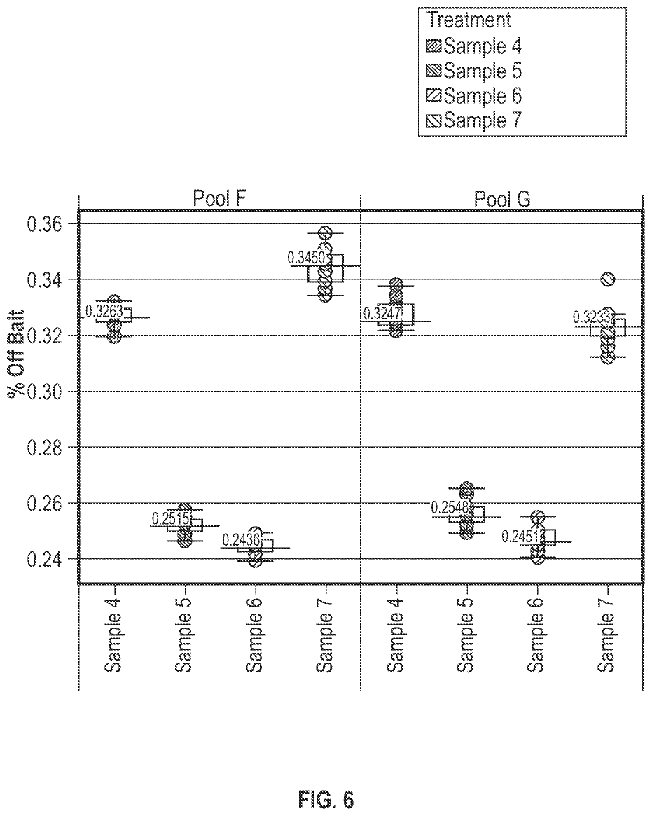

The Samples 4 to 7 were prepared as described with reference to Example 1 above. Sample 7 included 20× Cot-1 DNA but also included half the normal amount of the adapter blocking reagent. The percent off-bait capture from these Samples is shown in FIG. 6. In Library Pool F, and in Library Pool G, the samples using significantly more Cot-1 DNA (e.g., Sample 5 and Sample 6) performed best.

### Example 3. Off-Bait Capture with Diluted Cot-1 DNA

In Example 3, Samples were prepared with titrated down Cot-1 DNA. The Samples are summarized in Table 3 below:

TABLE 3
Samples with diluted Cot-1 DNA.

| | Cot-1 DNA Amount |
|---|---|
| Sample 8 (Control) | 100 μg |
| Sample 9 | 75 μg |
| Sample 10 | 50 μg |

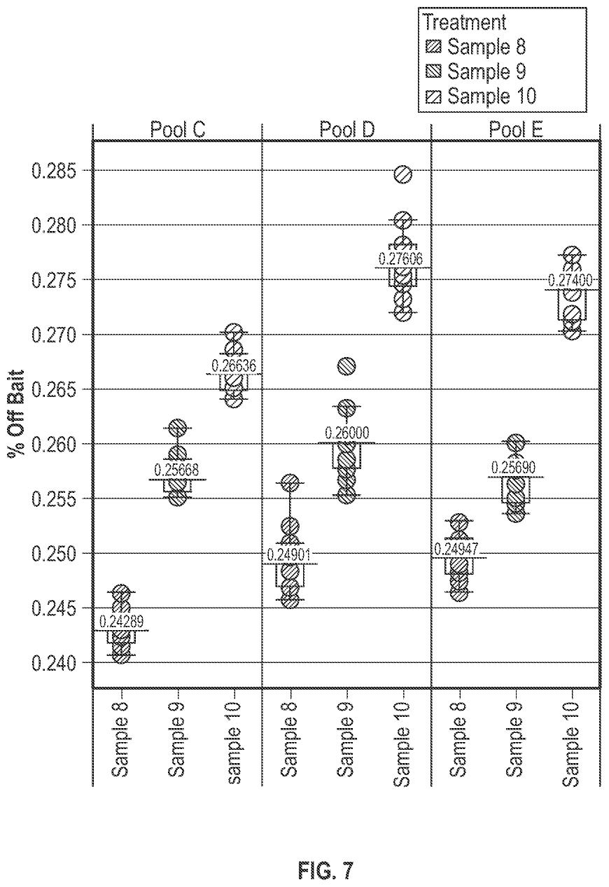

The resulting off-bait capture for each of these Samples in Library Pools C, D, and E are depicted in FIG. 7. The un-titrated 100 μg sample performed the best.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed may be resorted to by those of ordinary skill in the art, and that such modifications and variations are considered to be within the scope of examples of the present disclosure.

### Additional Examples

In some aspects, the techniques described herein relate to a method of processing genetic material for bioinformatic analysis, the method including: receiving a library of genetic material sourced from a sample; adding human Cot-1 DNA to the library at a ratio of between 100:1 and 20:1 of the human Cot-1 DNA to the genetic material to produce an enrichment pool; enriching the genetic material in the enrichment pool to produce enriched genetic material; and sequencing the enriched genetic material to produce sequencing data.

In some aspects, the techniques described herein relate to a method, wherein adding human Cot-1 DNA to the library is done at a ratio of 33:1.

In some aspects, the techniques described herein relate to a method, wherein receiving the library of genetic material includes receiving the input library with a mass of between 1 and 5 μg.

In some aspects, the techniques described herein relate to a method, wherein adding human Cot-1 DNA to the input library includes adding human Cot-1 DNA at a concentration of more than 8 mg/mL.

In some aspects, the techniques described herein relate to a method, wherein adding human Cot-1 DNA to the input library is done at a ratio of 33:1 and at a concentration of more than 8 mg/mL, and wherein receiving the input library of genetic material includes receiving the input library with a mass of between 1 and 5 μg.

In some aspects, the techniques described herein relate to a method, wherein enriching the genetic material produces at least 70% sequences captured by probes that constitute a portion of an exome of a subject that provided the sample.

In some aspects, the techniques described herein relate to a method, wherein enriching the genetic material produces less than 30% sequences captured by probes that do not constitute a portion of an exome of a subject that provided the sample.

In some aspects, the techniques described herein relate to a method, wherein sequencing of the enriched genetic material includes applying the enriched genetic material to one or more flow cells of a sequencing device.

In some aspects, the techniques described herein relate to a method, wherein the flow cells capture 50% to 70% more samples of the enriched genetic material with the added human Cot-1 DNA compared to enrichment pools prepared without the ratio of human Cot-1 DNA to the genetic material.

In some aspects, the techniques described herein relate to a method, wherein receiving of the library includes receiving two or more pooled libraries that have been combined.

In some aspects, the techniques described herein relate to a method, further including combining the two or more libraries.

In some aspects, the techniques described herein relate to a method, wherein receiving the library includes receiving ten or more pooled libraries that have been combined.

In some aspects, the techniques described herein relate to a method, wherein receiving the library includes receiving ten to fifty pooled libraries that have been combined.

In some aspects, the techniques described herein relate to a method, further including adding one or more additional blockers to the enrichment pool.

In some aspects, the techniques described herein relate to a method, wherein enriching the genetic material in the enrichment pool includes amplifying the genetic material prior to sequencing.

In some aspects, the techniques described herein relate to a method, wherein enriching the genetic material in the enrichment pool includes controlling concentration of the genetic material.

In some aspects, the techniques described herein relate to a method of processing genetic material for bioinformatic analysis, the method including: receiving a library of genetic material sourced from a sample; adding human Cot-1 DNA to the library to produce an enrichment pool, wherein the human Cot-1 DNA is at least 10 μL of human Cot-1 DNA a concentration of at least 10 mg/mL; enriching the genetic material in the enrichment pool to produce enriched genetic material; and sequencing the enriched genetic material to produce sequencing data.

In some aspects, the techniques described herein relate to a method, wherein adding human Cot-1 DNA to the library is done at a concentration of at least 12 mg/mL.

In some aspects, the techniques described herein relate to a method of processing genetic material for bioinformatic analysis, the method including: receiving a library of genetic material sourced from a sample; adding at least 100 μg human Cot-1 DNA to the library to produce an enrichment pool; enriching the genetic material in the enrichment pool to produce enriched genetic material; and sequencing the enriched genetic material to produce sequencing data.

In some aspects, the techniques described herein relate to a method, wherein receiving the library of genetic material includes receiving the library with a mass between 1 and 5 μg.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the claimed subject matter should be determined with reference to the appended claims, along with the full scope of equivalents to which such claims are entitled.

What is claimed is:

1. A method of processing genetic material for bioinformatic analysis, the method comprising:
receiving a library of genetic material sourced from a sample;
adding human Cot-1 DNA to the library at a ratio of between 100:1 and 20:1 of the human Cot-1 DNA to the genetic material to produce an enrichment pool;
enriching the genetic material in the enrichment pool to produce enriched genetic material; and
sequencing the enriched genetic material to produce sequencing data.

2. The method of claim 1, wherein adding human Cot-1 DNA to the library is done at a ratio of 33:1.

3. The method of claim 1, wherein receiving the library of genetic material comprises receiving the input library with a mass of between 1 and 5 μg.

4. The method of claim 1, wherein adding human Cot-1 DNA to the input library comprises adding human Cot-1 DNA at a concentration of more than 8 mg/mL.

5. The method of claim 1, wherein adding human Cot-1 DNA to the input library is done at a ratio of 33:1 and at a concentration of more than 8 mg/mL, and wherein receiving the input library of genetic material comprises receiving the input library with a mass of between 1 and 5 μg.

6. The method of claim 1, wherein enriching the genetic material produces at least 70% sequences captured by probes that constitute a portion of an exome of a subject that provided the sample.

7. The method of claim 1, wherein enriching the genetic material produces less than 30% sequences captured by probes that do not constitute a portion of an exome of a subject that provided the sample.

8. The method of claim 1, wherein sequencing of the enriched genetic material comprises applying the enriched genetic material to one or more flow cells of a sequencing device.

9. The method of claim 8, wherein the flow cells capture 50% to 70% more samples of the enriched genetic material with the added human Cot-1 DNA compared to enrichment pools prepared without the ratio of human Cot-1 DNA to the genetic material.

10. The method of claim 1, wherein receiving of the library comprises receiving two or more pooled libraries that have been combined.

11. The method of claim 10, further comprising combining the two or more libraries.

12. The method of claim 1, wherein receiving the library comprises receiving ten or more pooled libraries that have been combined.

13. The method of claim 1, wherein receiving the library comprises receiving ten to fifty pooled libraries that have been combined.

14. The method of claim 1, further comprising adding one or more additional blockers to the enrichment pool.

15. The method of claim 1, wherein enriching the genetic material in the enrichment pool comprises amplifying the genetic material prior to sequencing.

16. The method of claim 1, wherein enriching the genetic material in the enrichment pool comprises controlling concentration of the genetic material.

17. A method of processing genetic material for bioinformatic analysis, the method comprising:
receiving a library of genetic material sourced from a sample;
adding human Cot-1 DNA to the library to produce an enrichment pool, wherein the human Cot-1 DNA is at least 10 μL of human Cot-1 DNA a concentration of at least 10 mg/ml;
enriching the genetic material in the enrichment pool to produce enriched genetic material; and
sequencing the enriched genetic material to produce sequencing data.

18. The method of claim 17, wherein adding human Cot-1 DNA to the library is done at a concentration of at least 12 mg/mL.

19. A method of processing genetic material for bioinformatic analysis, the method comprising:
receiving a library of genetic material sourced from a sample;
adding at least 100 μg human Cot-1 DNA to the library to produce an enrichment pool;
enriching the genetic material in the enrichment pool to produce enriched genetic material; and
sequencing the enriched genetic material to produce sequencing data.

20. The method of claim 19, wherein receiving the library of genetic material comprises receiving the library with a mass between 1 and 5 μg.
